(19) United States
(12) Patent Application Publication
(10) Pub. No.: US 2024/0299424 A1
(43) Pub. Date: Sep. 12, 2024

(54) USE OF PHYTOCANNABINOIDS FOR TREATING ENDOMETRIAL CANCER AND ENDOMETRIOSIS

(71) Applicant: INTEGRATIVE THERAPY DISCOVERY LAB S.R.L., Camerino (IT)

(72) Inventors: Massimo NABISSI, San Ginesio (IT); Oliviero MARINELLI, Colonnella (IT); Cristina AGUZZI, Falconara Marittima (IT); Laura ZEPPA, Camerino (IT); Maria Beatrice MORELLI, Montegranaro (IT)

(21) Appl. No.: 18/578,536

(22) PCT Filed: Jul. 12, 2022
(86) PCT No.: PCT/US2022/036758

§ 371 Date: Jan. 11, 2024

Related U.S. Application Data
(60) Provisional application No. 63/220,938, filed on Jul. 12, 2021.

(51) Int. Cl. A61K 31/00; A61P 35/00

(52) U.S. Cl. CPC A61K 31/658; A61P 35/00

(57) ABSTRACT

The present disclosure relates to the use of suitable phytocannabinoids such as cannabidivarin, cannabinol, cannabigerol, cannabidiol, delta9-tetrahydrocannabinol, cannabichromene, or combinations with or without cannabidiol thereof for treatment endometrial cancer and endometriosis and/or similar conditions, diseases, or disorders.

### CROSS REFERENCE TO RELATED APPLICATIONS

This application claims priority to U.S. 63/220,938 filed Jul. 12, 2021. The contents of this application are incorporated by reference in their entirety.

### FIELD OF THE INVENTION

The present disclosure relates to the use of phytocannabinoids for treatment of endometrial cancer, for example, the use of cannabidivarin (CBDV), cannabinol (CBN), cannabigerol (CBG), cannabichromene (CBC), delta9-tetrahydrocannabinol (THC) or combinations thereof.

The present disclosure relates to the use of phytocannabinoids for treatment of endometriosis, for example, the use of cannabidiol (CBD), CBDV, CBN, CBG, CBC, THC or combinations thereof.

The present disclosure relates to the use of phytocannabinoids for treatment of solid cancers, for example, the use of CBD, CBDV, CBN, CBG, CBC, THC or combinations thereof.

### BACKGROUND OF THE INVENTION

### Endometrial Cancer

Endometrial cancer (EC) is the most commonly diagnosed gynecological malignancy in developed countries. ECs are divided into two subtypes, type I endometrioid EC and type II non-endometrioid EC. Type I is well differentiated and is frequently associated with a hyper estrogenic environment. Type II carcinomas develop from atrophic endometrium and are classified in different histological subgroups (e.g., serous and clear cell adenocarcinomas). These subtypes are poorly differentiated and associated with dismal prognosis [1, 2]. Treatment options are hysterectomy, chemotherapy, such as triplet therapy with paclitaxel, cisplatin, and doxorubicin, and combinations with radiation, but only limited options remain if the tumors relapse or metastasize [3]. One study has showed a potential anti-tumor role only for cannabidiol, by means of modulating cell signaling pathways involved. For example, cannabidiol reduces cell viability, activating predominantly cell death in type I cells and autophagy in mixed type endometrial cancer cell lines and improves chemotherapeutic drugs cytotoxicity [4], while no data are available for other phytocannabinoids.

### Endometriosis

Endometriosis is estimated to affect 10% of reproductive-age women, which extrapolates to approximately 190 million women worldwide, given the World Bank's population valuations for 2019 [5, 6]. The true prevalence of endometriosis is uncertain, however, because definitive diagnosis requires surgical visualization. Estimates vary widely among population samples and diagnostic approaches [7]. The prevalence ranges from 2 to 11% among asymptomatic women, 5 to 50% among infertile women, and 5 to 21% among women hospitalized for pelvic pain. Among symptomatic adolescents, the prevalence of endometriosis ranges from 49% for those with chronic pelvic pain to 75% for those with pain that is unresponsive to medical treatment [5]. Inflammatory dysregulation plays a vital role in endometriosis's pathogenesis [8]. Inflammatory cytokines are elevated in endometriosis lesions and peritoneal fluid of women with endometriosis [8]. Specifically, elevated IL-8 has been shown in the peritoneal fluid of women with endometriosis [9], and IL-6, in the peritoneal fluid, has been linked to infertility in women with endometriosis [10]. Thus, the reversion of inflammatory dysregulation is a necessary characteristic for a robust therapeutical approach.

In addition, one of the most important pathogenic characteristics of the proliferation of endometriosis, especially in the deep-infiltrating form, is the invasion of endometriotic cells through the basilar membrane of the peritoneal mesothelium into the extracellular matrix [11].

So, a potential therapeutical approach to reduce endometriosis cells inflammatory state and migration could have beneficial effects for endometriosis.

Endometriosis is still considered incurable and the discovery of new treatments to improve the currently available therapies remains desirable.

### SUMMARY OF THE INVENTION

It is an object of the present invention to provide methods and compositions using phytocannabinoids to treat endometrial cancers, other solid cancers and endometriosis.

It is an object of the present invention to provide methods and compositions using CBG, CBC, CBN, CBDV, THC alone and in combination and the combination of each single phytocannabinoid with chemotherapeutic drugs (paclitaxel, cisplatin and doxorubicin) or CBD to treat endometrial cancer.

It is an object of the present invention to provide methods and compositions using CBD, CBG, CBC, CBN, CBDV, THC alone and in combination and the combination of each single phytocannabinoid to treat endometriosis.

### Endometrial Cancer

Accordingly, the present disclosure includes a method of treating endometrial cancer in a subject in need thereof, comprising administering an effective amount of at least one phytocannabinoid to the subject, with the proviso that for single administration the at least one phytocannabinoid does not comprise cannabidiol.

The present disclosure also includes a use of an effective amount of at least one phytocannabinoid for treatment of an endometrial cancer in a subject in need thereof, with the proviso that for single administration the at least one phytocannabinoid does not comprise cannabidiol.

The present disclosure also includes a use of an effective amount of at least one phytocannabinoid for preparation of a medicament for treatment of an endometrial cancer in a subject in need thereof, with the proviso that for single administration the at least one phytocannabinoid does not comprise cannabidiol.

The present disclosure also includes at least one phytocannabinoid to treat an endometrial cancer in a subject in need thereof, with the proviso that for single administration the at least one phytocannabinoid does not comprise cannabidiol.

In an embodiment, the at least one phytocannabinoid comprises cannabidiol (not as single treatment), cannabidivarin, cannabinol, cannabigerol, cannabichromene, delta9-tetrahydrocannabinol or combinations thereof.

In another embodiment of the present disclosure, the at least one phytocannabinoid is cannabidiol (not as single treatment), cannabidivarin, cannabinol, cannabigerol, cannabichromene, delta9-tetrahydrocannabinol or combinations thereof.

In an embodiment, the at least one phytocannabinoid is cannabidivarin.

In another embodiment, the at least one phytocannabinoid is cannabinol.

In another embodiment, the at least one phytocannabinoid is cannabigerol.

In another embodiment, the at least one phytocannabinoid is cannabichromene.

In another embodiment, the at least one phytocannabinoid is delta9-tetrahydrocannabinol

In an embodiment, the combination of at least one phytocannabinoid that is cannabidivarin, cannabinol, cannabigerol or cannabichromene, delta9-tetrahydrocannabinol is a combination comprising cannabidiol.

In another embodiment, the combination further consists of cannabigerol.

In a further embodiment, the combination further consists of cannabichromene.

In another embodiment, the combination further consists of cannabidivarin.

In another embodiment, the combination further consists of delta9-tetrahydrocannabinol.

In an embodiment, the neoplasm is endometrial cancer.

In an embodiment, the at least one phytocannabinoid is administered or for use in combination with at least one other anticancer treatment, for example a chemotherapeutical drug.

In an embodiment, the subject is a human.

### Endometriosis

Other objects of the invention are achieved by providing a method of treating endometriosis in a subject in need thereof, comprising administering an effective amount of at least one phytocannabinoid to the subject.

The present disclosure also includes a use of an effective amount of at least one phytocannabinoid for treatment of endometriosis in a subject in need thereof.

The present disclosure also includes a use of an effective amount of at least one phytocannabinoid for preparation of a medicament for treatment of endometriosis in a subject in need thereof.

The present disclosure also includes at least one phytocannabinoid for use to treat endometriosis in a subject in need thereof.

In an embodiment, the at least one phytocannabinoid comprises cannabidiol, cannabidivarin, cannabinol, cannabigerol, cannabichromene, delta9-tetrahydrocannabinol or combinations thereof.

In another embodiment of the present disclosure, the at least one phytocannabinoid is cannabidiol, cannabidivarin, cannabinol, cannabigerol, cannabichromene, delta9-tetrahydrocannabinol or combinations thereof.

In an embodiment, the at least one phytocannabinoid is cannabidivarin.

In another embodiment, the at least one phytocannabinoid is cannabinol.

In another embodiment, the at least one phytocannabinoid is cannabigerol.

In another embodiment, the at least one phytocannabinoid is cannabichromene.

In another embodiment, the at least one phytocannabinoid is delta9-Tetrahydrocannabinol.

In another embodiment, the at least one phytocannabinoid is cannabidiol.

In an embodiment, the at least one phytocannabinoid is cannabidiol alone and without any other phytocannabinoids.

In an embodiment, the combination of at least one phytocannabinoid that is cannabidivarin, cannabinol, cannabigerol, cannabichromene, delta9-tetrahydrocannabinol is a combination comprising cannabidiol.

In another embodiment, the combination further consists of cannabigerol.

In a further embodiment, the combination further consists of cannabichromene.

In another embodiment, the combination further consists of cannabidivarin.

In another embodiment, the combination further consists of delta9-tetrahydrocannabinol

In an embodiment, the at least one phytocannabinoid is administered with at least one endometriosis treatment.

In an embodiment, the subject is a human.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should rather be given the broadest interpretation consistent with the description as a whole.

### BRIEF DESCRIPTION OF THE DRAWINGS

The embodiments of the disclosure will now be described in greater detail with reference to the attached drawings, in which:

FIGS. 1,3,4 are plots of cell viability (% vs vehicle) as a function of dose (microM) showing that phytocannabinoids cannabigerol (CBG), cannabichromene (CBC), cannabinol (CBN), cannabidivarin (CBDV) and delta9-tetrahydrocannabinol (THC) induced cytotoxicity in endometrial cancer cell line Ishikawa. Cell line was treated for 72 hours with different doses of phytocannabinoid alone (FIG. 1) or in double with cannabidiol (10, 15 and 20 microM CBD; FIG. 3-4), as indicated on the plots. Cell viability was determined by MTT assay. Data shown are expressed as mean±SD of three separate experiments. * p<0.05 versus non-treated cells. PRISM 5.0a software was used for statistical analysis.

FIG. 2 is a chart of the specific IC₅₀ for each tested compound determined in Ishikawa cell lines. IC₅₀ was calculated by PRISM 5.0a software, for single phytocannabinoids.

FIG. 5 is a plot of cell viability (% vs vehicle) as a function of dose (microM) showing that phytocannabinoids cannabigerol (CBG), cannabichromene (CBC), cannabinol (CBN), cannabidivarin (CBDV) and delta9-tetrahydrocannabinol (THC) induced cytotoxicity in endometrial cancer cell line PCEM004A (according to exemplary embodiments of the present disclosure). Cell line was treated for 72 hours with different doses of phytocannabinoid alone. Cell viability was determined by MTT assay. Data shown are expressed as mean±SD of three separate experiments. * p<0.05 versus non-treated cells. PRISM 5.0a software was used for statistical analysis.

FIG. 6 is a chart of the specific IC₅₀ for each tested compound determined in PCEM004A cell lines. IC₅₀ was calculated by PRISM 5.0a software, for single phytocannabinoids.

FIGS. 7-10 are plots of cell viability (% vs vehicle) as a function of dose (microM) showing that phytocannabinoids cannabigerol (CBG), cannabichromene (CBC), cannabinol (CBN), cannabidivarin (CBDV) and delta9-tetrahydrocannabinol (THC) in combination with paclitaxel (PAC), doxorubicin (DOX) and cisplatin (CIS) induced cytotoxicity in endometrial cancer cell lines Ishikawa and PCEM004A. It was found that phytocannabinoids (CNBDs) improve chemotherapeutic drug effects in Ishikawa and PCEM004A cell lines. Cell viability was determined by MTT assay. Cells were treated for 72 hours with CBG, CBC, CBN, CBDV and THC, alone and in combination with different doses of CIS, DOX and PAC. Data shown are expressed as mean±SD of three separate experiments. * p<0.05 versus CNBDs alone, #p<0.05 versus chemotherapeutic drug alone.

FIGS. 11,13 are plots of cell viability (% vs vehicle) as a function of dose (microM) showing that phytocannabinoids cannabigerol (CBG), cannabichromene (CBC), cannabinol (CBN), cannabidivarin (CBDV) and delta9-tetrahydrocannabinol (THC) induced cytotoxicity in pancreatic ductal adenocarcinoma cancer cell line PANC-1 and in glioblastoma cell line U251. Cell viability was evaluated at 72 hours post-treatment, by MTT assay. Data shown are expressed as mean±SD of three separate experiments and six wells for each dose. * p<0.05 versus vehicle. It was found that CNBDs induce cytotoxicity in PANC-1 and U251 cell lines.

FIG. 12 is a chart of the specific IC₅₀ for each tested compound determined in PANC-1 cell line. IC₅₀ was calculated by PRISM 5.0a software, for single phytocannabinoids.

FIG. 14 is a chart of the specific IC₅₀ for each tested compound determined in U251 cell line. IC₅₀ was calculated by PRISM 5.0a software, for single phytocannabinoids.

### Endometriosis Data

FIGS. 15-20 are plots of cell viability (% vs vehicle) as a function of dose (microM) showing that phytocannabinoids cannabigerol (CBG), cannabichromene (CBC), cannabinol (CBN), cannabidiol (CBD), cannabidivarin (CBDV) and delta9-tetrahydrocannabinol (THC) induced cytotoxicity in 12Z endometriotic cell line (according to exemplary embodiments of the present disclosure). Cell line was treated with single administration of different doses of phytocannabinoids alone, as indicated on the plots. Cell viability was evaluated at 72 hours post-treatment, by MTT assay. Data shown are expressed as mean±SE of three separate experiments and six wells for each dose. * p<0.5. IC₅₀ was calculated by PRISM 5.0a software, for single phytocannabinoids.

FIG. 21 is a chart of the specific IC₅₀ for each tested compound determined in 12Z cell line. IC₅₀ was calculated by PRISM 5.0a software, for single phytocannabinoids.

FIGS. 22-29 are representative images showing HOECHST fluorescence after 24 hours 12Z culture in Transwell chambers, showing that phytocannabinoids cannabigerol (CBG), cannabichromene (CBC), cannabinol (CBN), cannabidiol (CBD), cannabidivarin (CBDV) and delta9-tetrahydrocannabinol (THC) affect invasion ability of 12Z endometriotic cell line. Cell line was treated with different doses of phytocannabinoids alone, as indicated on the figures.

FIG. 30 is a chart summarizing the results of FIGS. 22-29. Bar represent the quantification of invaded cells in each field. Error bars represent SD. *p<0.05.

FIG. 31-32 are plots of The TaqMan® Array—Well FAST Plate customed with the main inflammatory genes analyzed in 12Z endometriosis cell line. Gene expression (Normalized gene expression versus vehicle) as a function of dose showing that phytocannabinoids cannabigerol (CBG, 30 microM), cannabichromene (CBC, 30 microM), cannabinol (CBN, 30 microM), cannabidiol (CBD, 10 microM), cannabidivarin (CBDV, 10 microM) and delta9-tetrahydrocannabinol (THC, 40 microM) induced reduced gene expression of different cytokines in 12Z endometriotic cell line (according to exemplary embodiments of the present disclosure). Cell line was treated with single administration of each phytocannabinoid, as indicated on the plots. Gene expression was evaluated at 48 hours post-treatment, by RT/PCR array. Data shown are expressed as mean±SE of three separate experiments. * p<0.05 vs vehicle was calculated by 2^{-ΔΔCt} method.

### DETAILED DESCRIPTION OF THE INVENTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “cannabidiol” and the abbreviation “CBD” as used herein refers to the phytocannabinoid 2-[(1R,6R)-3-methyl-6-prop-1-en-2-ylcyclohex-2-en-1-yl]-5-pentylbenzene-1,3-diol of the structure:

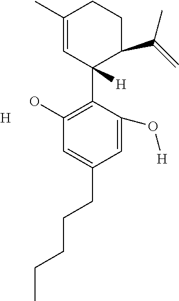

The term “cannabigerol” and the abbreviation “CBG” as used herein refers to the phytocannabinoid 2-[(2E)-3,7-dimethylocta-2,6-dienyl]-5-pentylbenzene-1,3-diol of the structure:

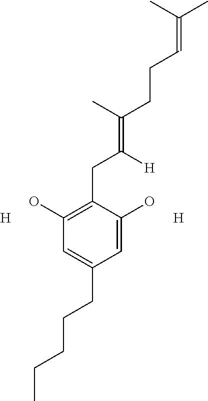

The term “cannabinol” and the abbreviation “CBN” as used herein refers to the phytocannabinoid 6,6,9-trimethyl-3-pentylbenzo[c]chromen-1-ol of the structure:

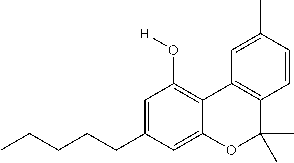 [SMILES: CCCCCc1cc(O)c2c(c1)OC(C)(C)c1ccc(C)cc1-2]

The term “cannabichromene” and the abbreviation “CBC” as used herein refers to the phytocannabinoid 2-methyl-2-(4-methylpent-3-enyl)-7-pentylchromen-5-ol of the structure:

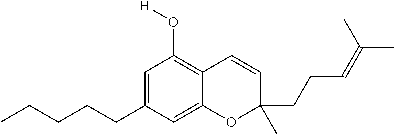 [SMILES: CCCCCc1cc(O)c2c(c1)OC(C)(CCC=C(C)C)C=C2]

The term “cannabidivarin” and the abbreviation “CBDV” as used herein refers to the phytocannabinoid 2-[(1R,6R)-3-methyl-6-prop-1-en-2-ylcyclohex-2-en-1-yl]-5-propylbenzene-1,3-diol of the structure:

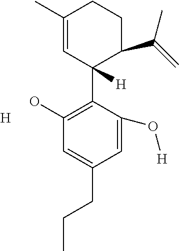

The term “delta9-tetrahydrocannabinol” and the abbreviation “THUC” as used herein refers to the phytocannabinoid (6aR,10aR)-6,6,9-trimethyl-3-pentyl-6a,7,8,10a-tetrahydrobenzo[c]chromen-1-ol of the structure:

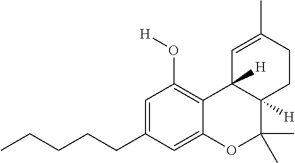

The term “subject” as used herein includes all members of the animal kingdom including mammals. In an embodiment, the subject is a human.

The term “pharmaceutically acceptable” means compatible with the treatment of subjects, for example, mammals such as humans.

The term “enteral” as used herein means taken into the body or administered or used in a manner that is through the gastrointestinal tract.

The term “parenteral” as used herein means taken into the body or administered or used in a manner other than through the gastrointestinal tract.

The terms “to treat”, “treating”, “treatment” and the like as used herein and as is well understood in the art, refer to an approach for obtaining beneficial or desired results, including clinical results. Beneficial or desired results include, but are not limited to, alleviation or amelioration of one or more symptoms of a disease, condition or disorder such as endometrial cancer, diminishment of the extent of the disease, condition or related disorders, stabilized (i.e. not worsening) disease, condition or disorder such as endometrial cancer, delay or slowing of the progression of the disease, condition or disorder such as endometrial cancer, amelioration or palliation of the state of the disease, condition or disorder such as endometrial cancer and/or remission (whether partial or total) of the disease, condition or disorder such as endometrial cancer, whether detectable or undetectable. “To treat”, “treating”, “treatment” and the like as used herein also include prophylactic treatment of the disease, condition or disorder such as endometrial cancer. For example, a subject with early stage endometrial cancer is treated to prevent or delay progression or alternatively a subject in remission is treated to prevent or delay recurrence.

As used herein, the term “effective amount” and the like as used herein means an amount effective, at dosages and for periods of time necessary to achieve a desired result. For example, in the context of treating endometrial cancer, an effective amount of the at least one phytocannabinoid is an amount that, for example, reduces the endometrial cancer compared to the endometrial cancer without administration of the at least one phytocannabinoid. By “reducing the endometrial cancer” it is meant, for example, reducing the number of endometrial cancer cells, reducing the symptoms of the endometrial cancer and/or slowing the advancement of the endometrial cancer. Effective amounts may vary according to factors such as the disease state, age, and/or weight of the subject. The amount of the at least one phytocannabinoid that will correspond to such an amount will vary depending upon various factors, such as the given phytocannabinoid or combination thereof, the pharmaceutical formulation, the route of administration or use, the identity of the subject being treated, and the like, but can nevertheless be routinely determined by one skilled in the art having reference to this disclosure.

### EXPERIMENTS

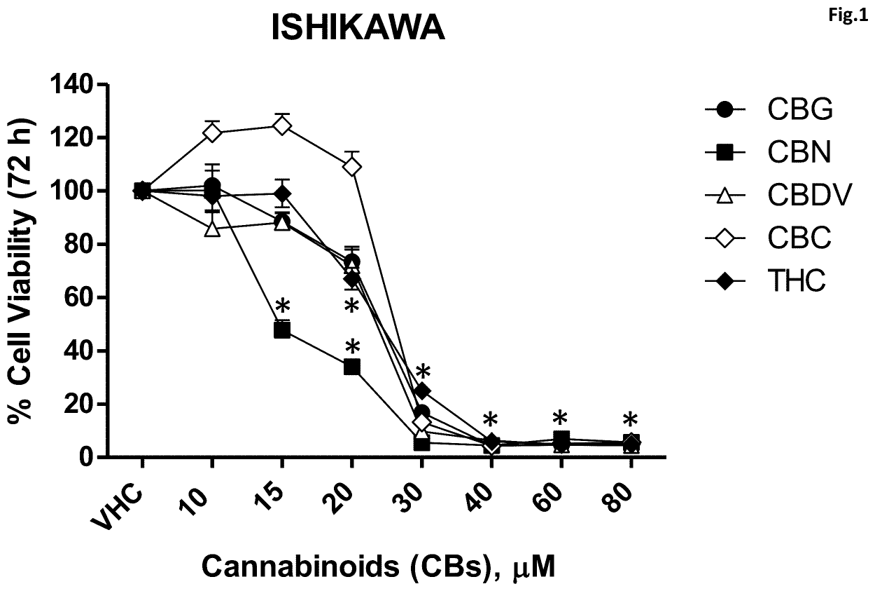

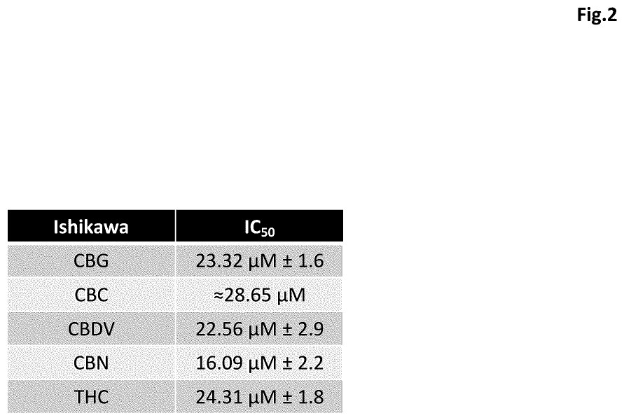

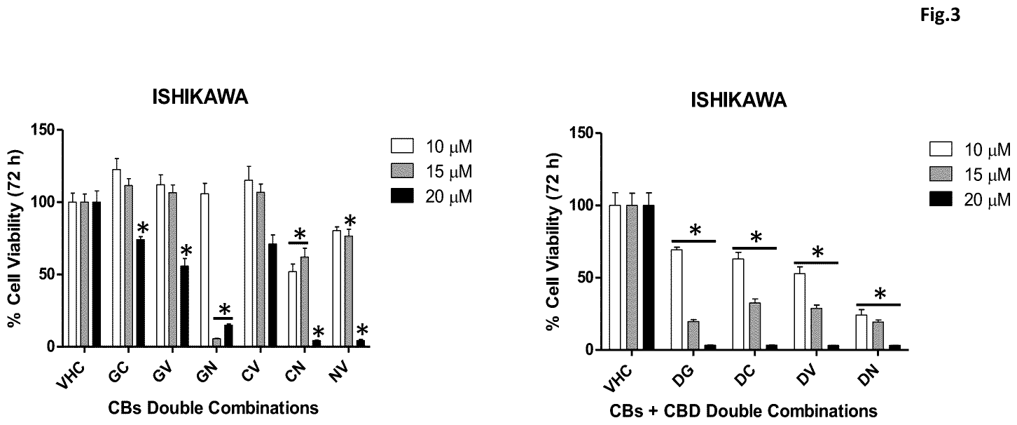

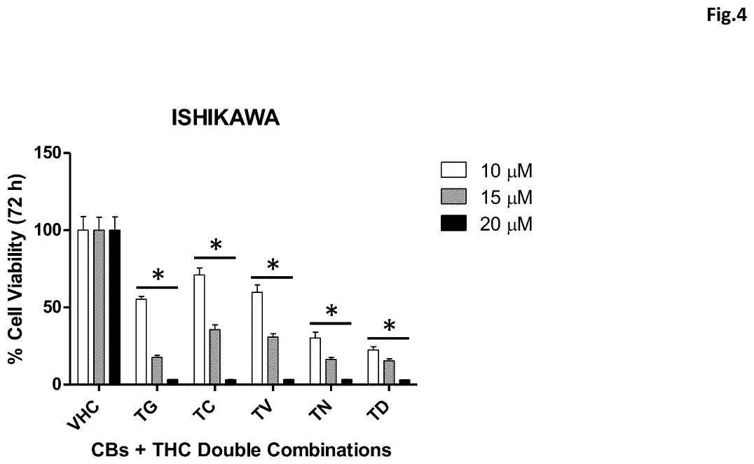

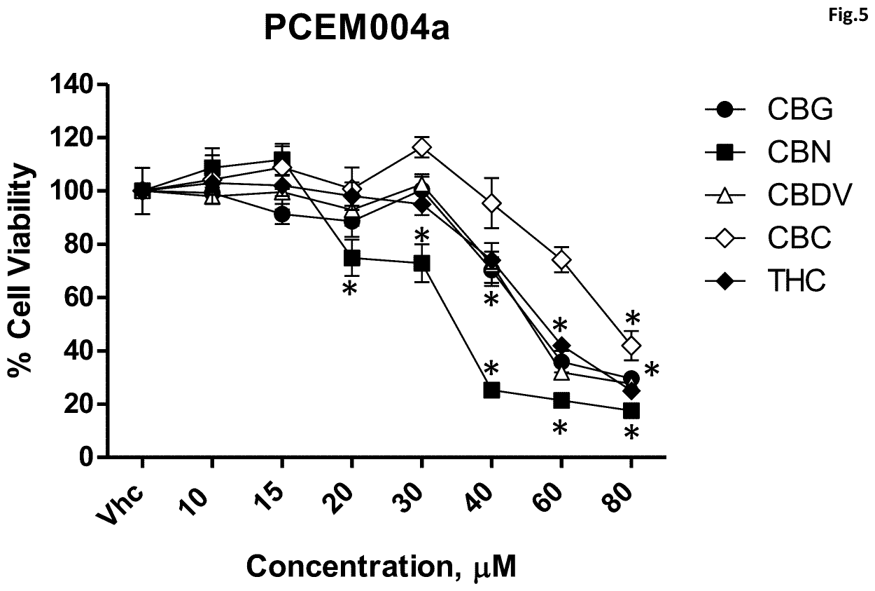

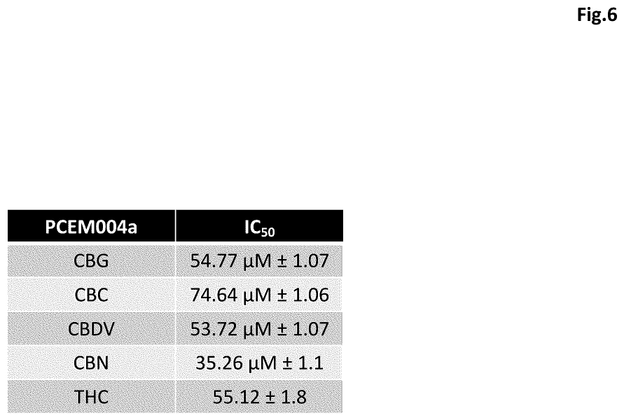

Through inventor experiments, it was evidenced that the phytocannabinoids cannabigerol (CBG), cannabinol (CBN), cannabichromene (CBC), cannabidivarin (CBDV) and delta-9 tetrahydrocannabinol (THC) alone (FIG. 1) with different potency (FIG. 2) and in combination (FIG. 3, 4) induced cytotoxicity in Ishikawa type I endometrial cancer cell line. In addition, the single phytocannabinoids were tested and found effective in one primary mixed type PCEM004A endometrial cancer cell line (FIG. 5) with different potency (FIG. 6). A dose dependent effect in the endometrial cancer cell lines tested was observed for all individual phytocannabinoids, with the efficacy in reducing cell viability generally highest for CBN, followed by CBDV, CBG, THC and CBC (FIG. 1, FIG. 2; FIG. 5, FIG. 6), with Ishikawa cells more sensitive than PCEM004A.

The results evidenced that most of the combinations were more effective than single cannabinoids and that CBN amplified the effect of all cannabinoids in reducing endometrial cancer cells viability in all treatments.

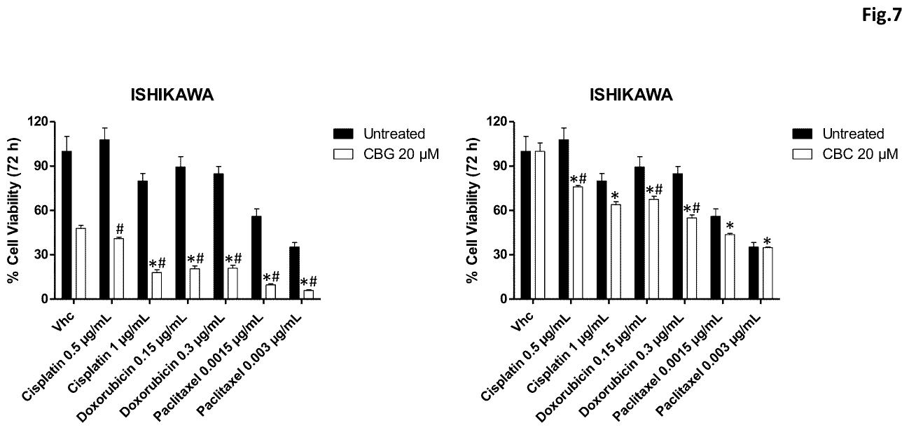

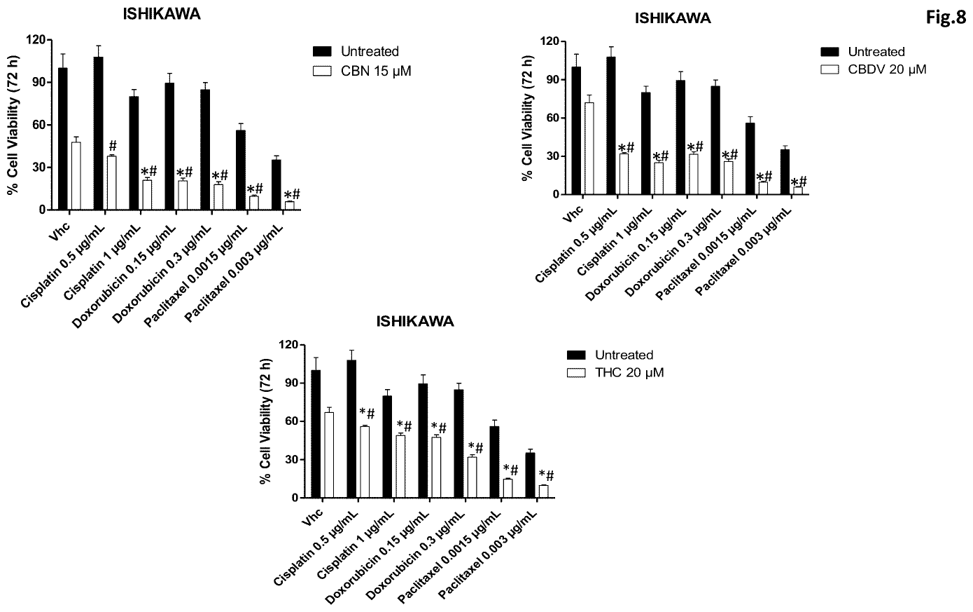

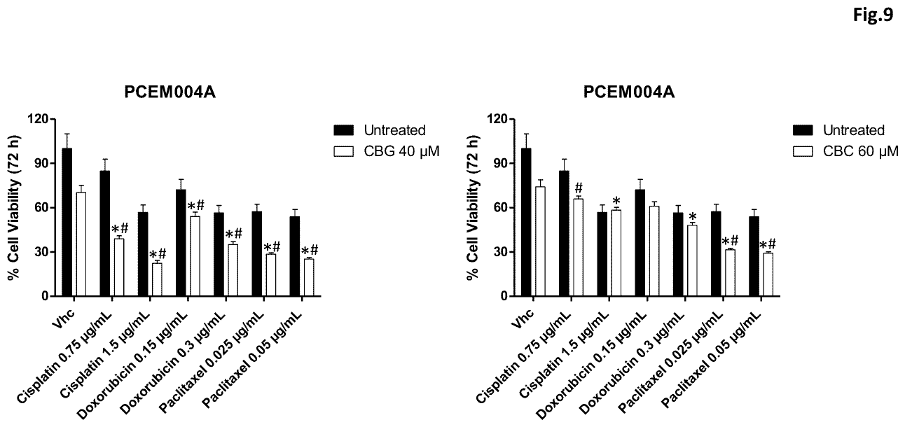

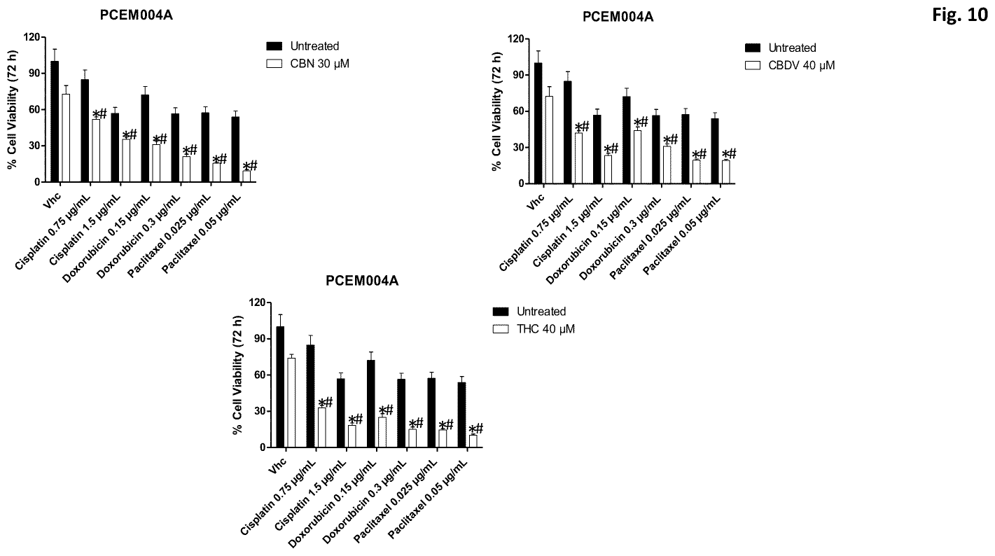

The effect of each phytocannabinoid was also evaluated in combination with the chemotherapeutic drugs used in the clinical practice for endometrial cancer. The results evidenced as the phytocannabinoids don't interfere with chemotherapeutic drugs effects and could improve their cytotoxic effects (FIG. 7-10), in both endometrial cancer cell lines.

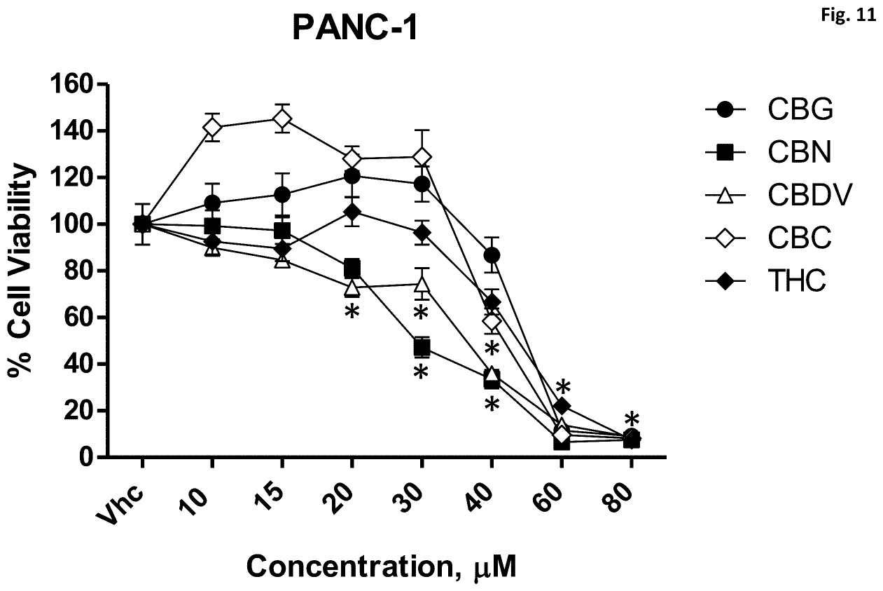

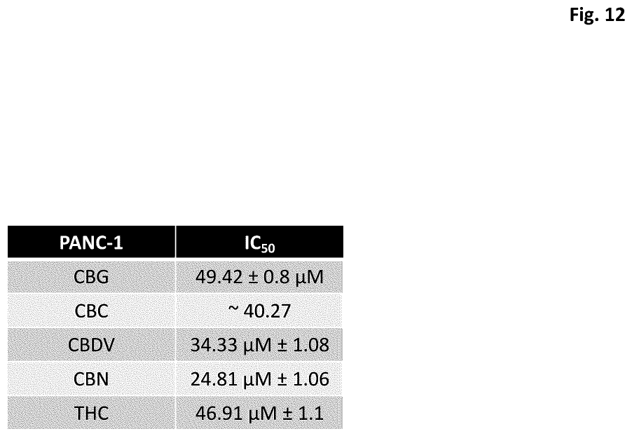

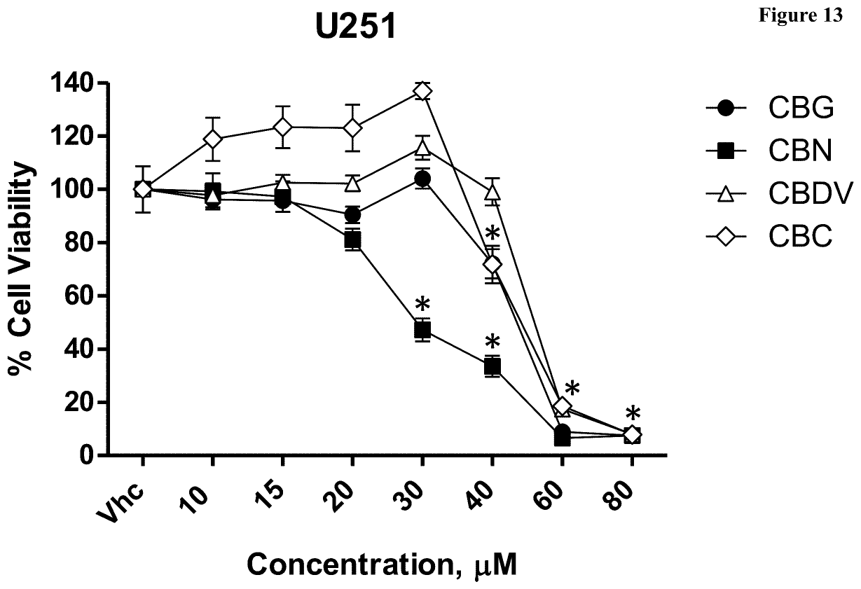

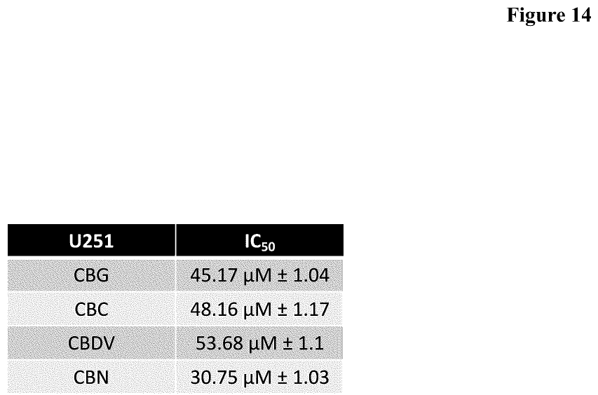

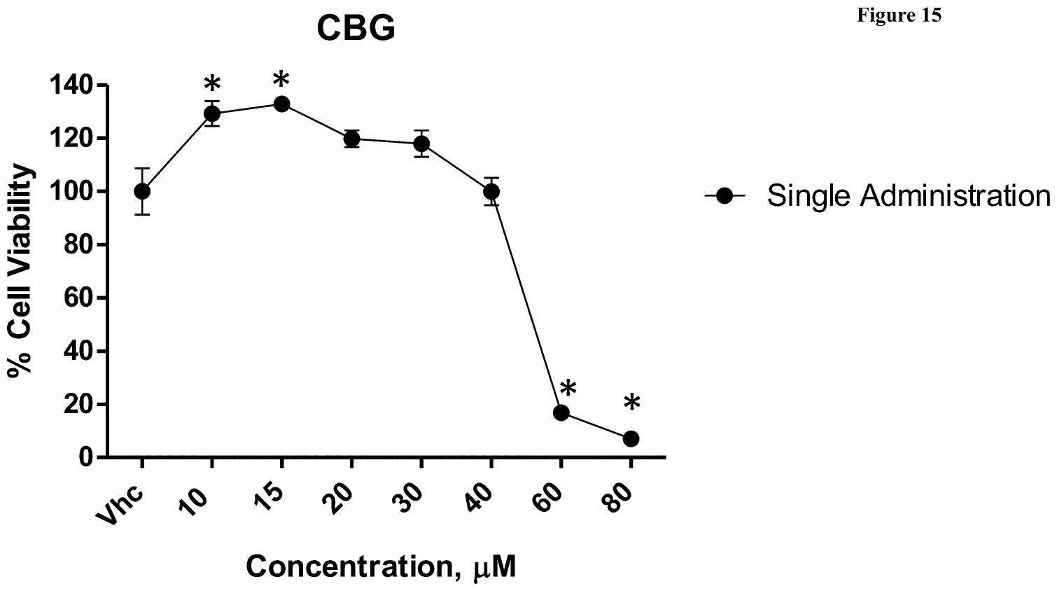

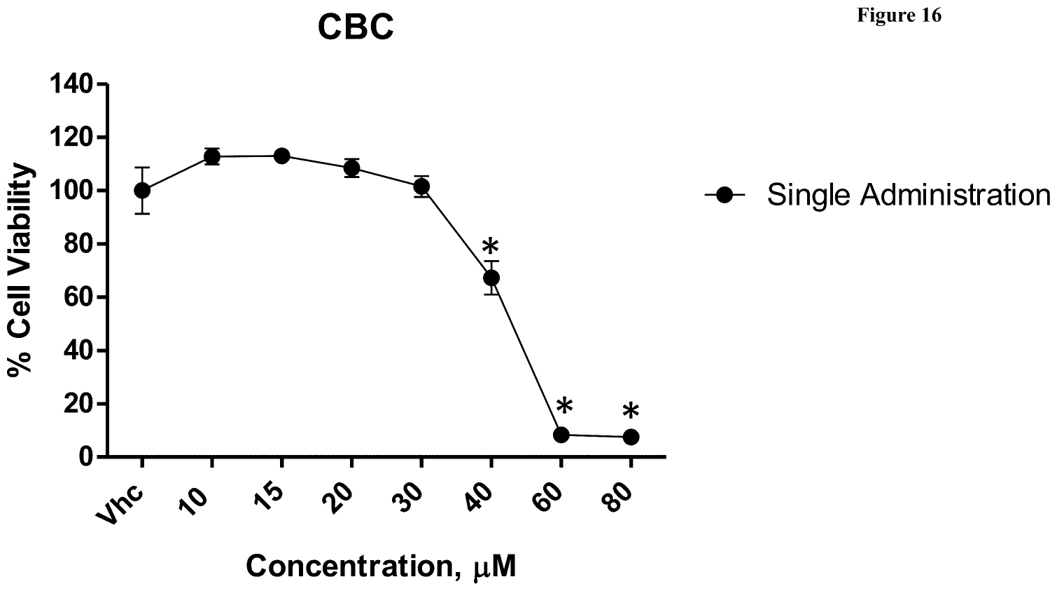

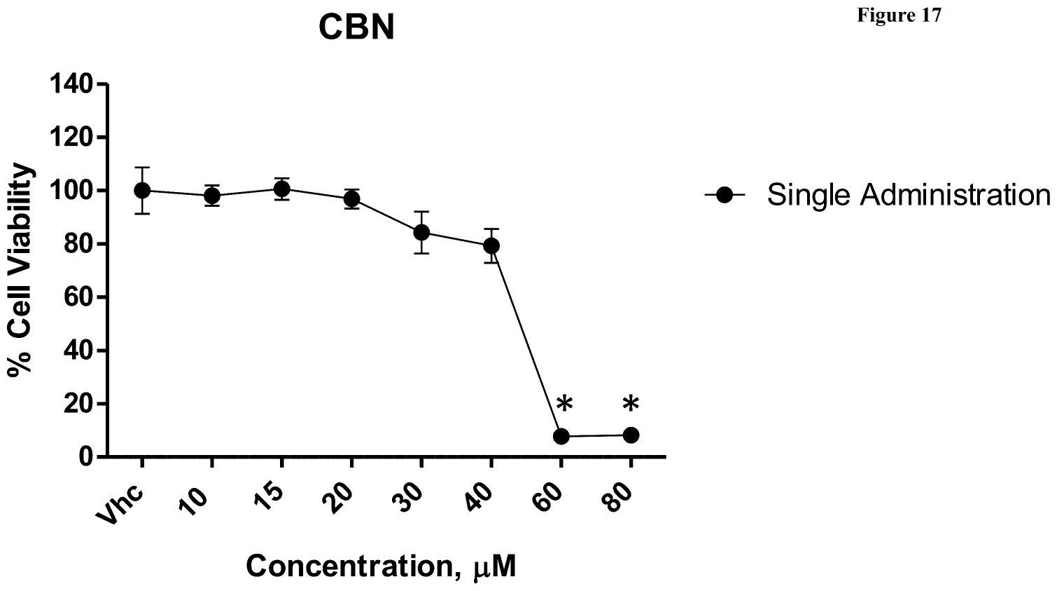

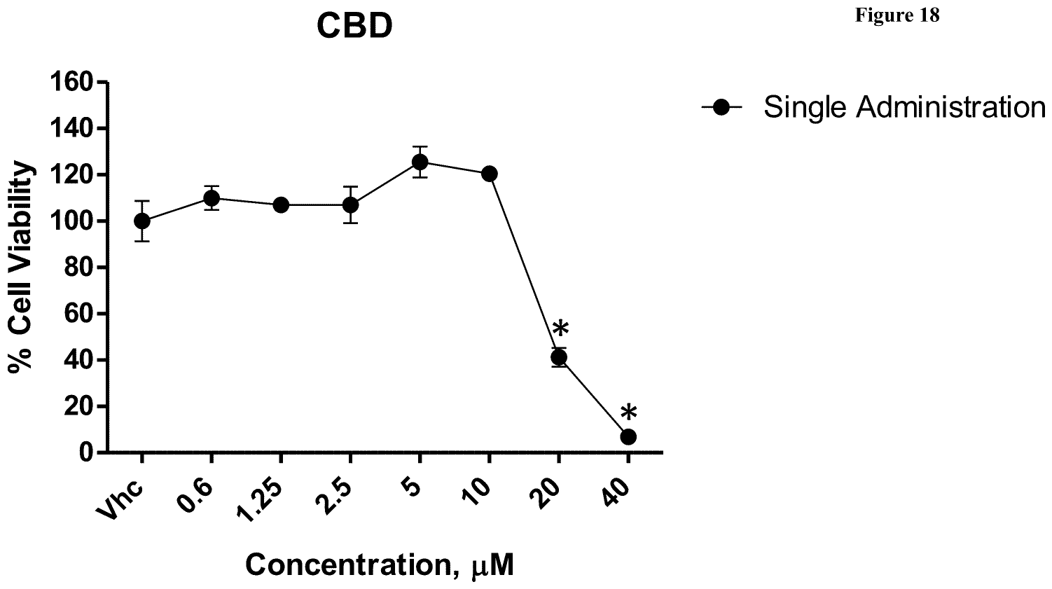

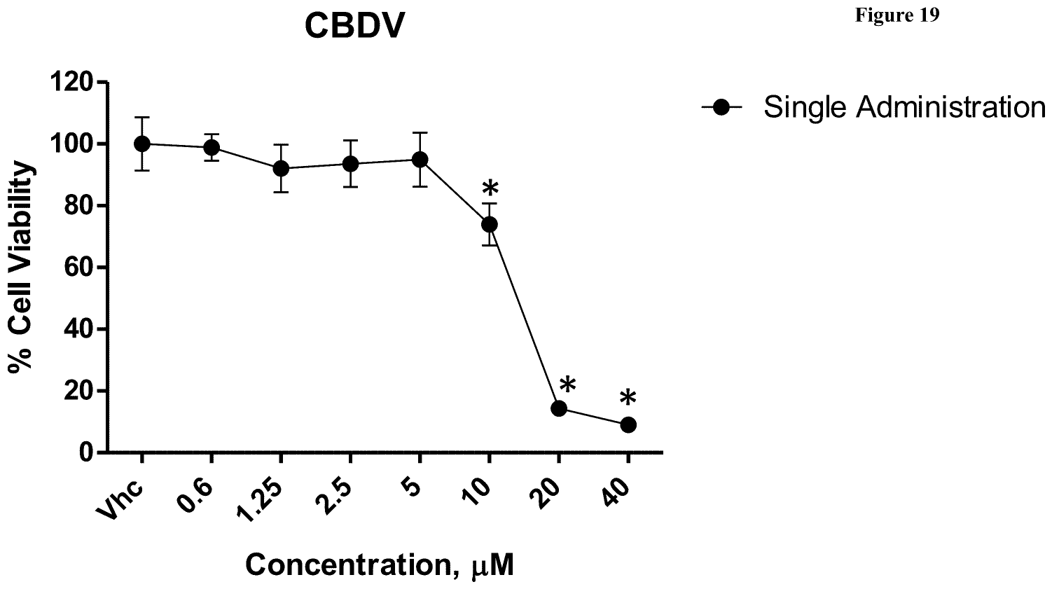

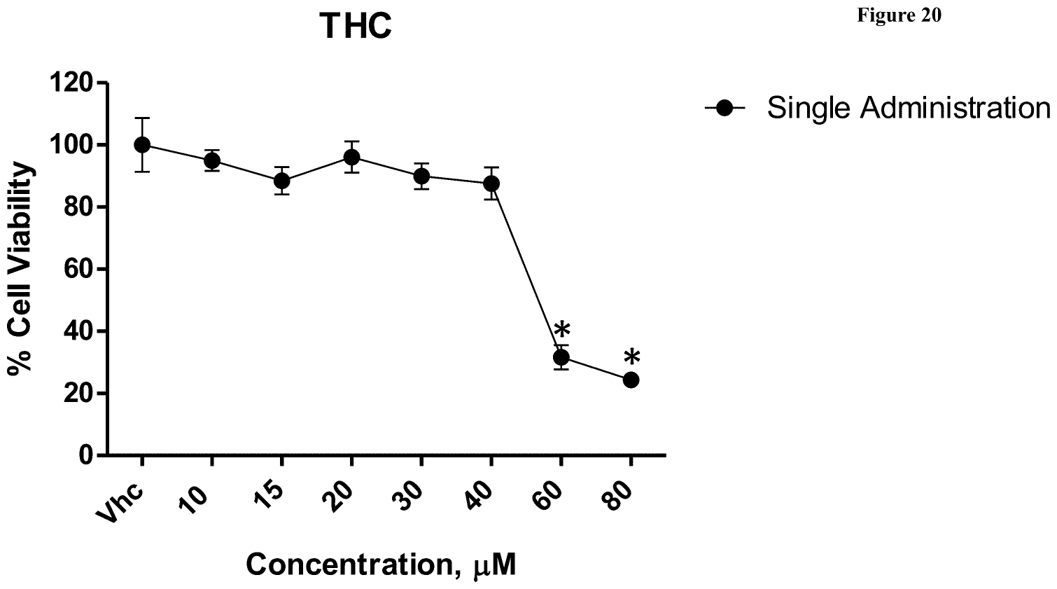

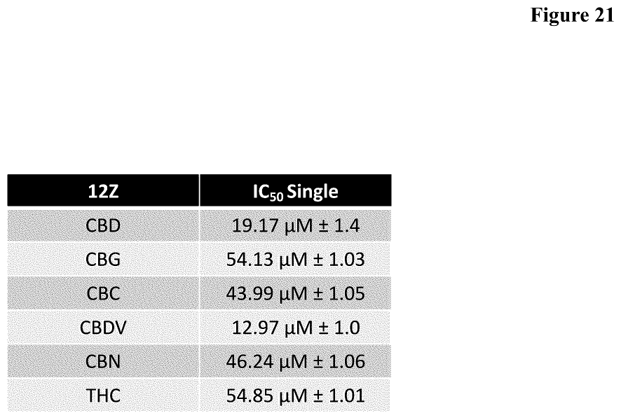

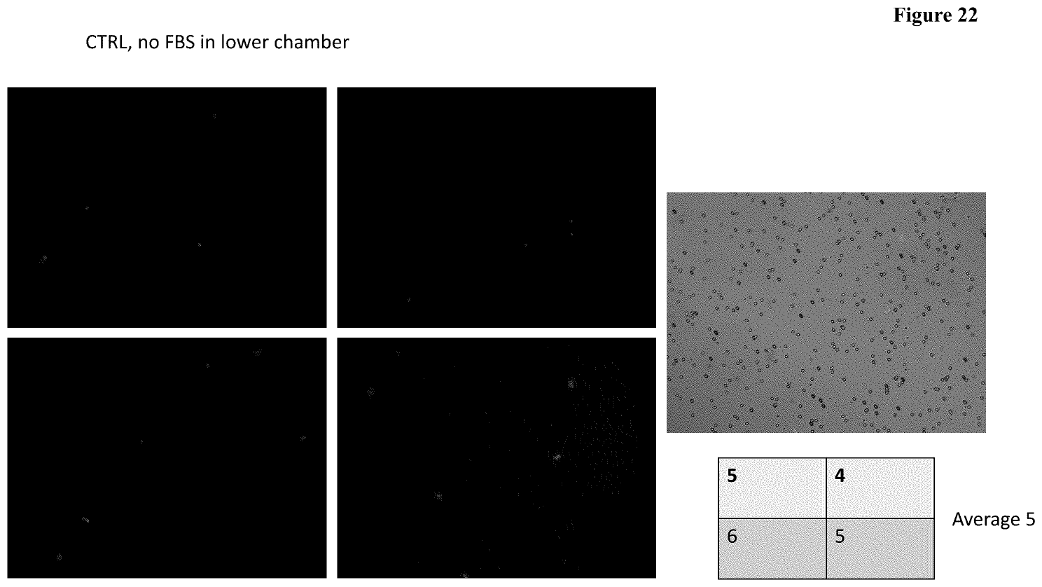

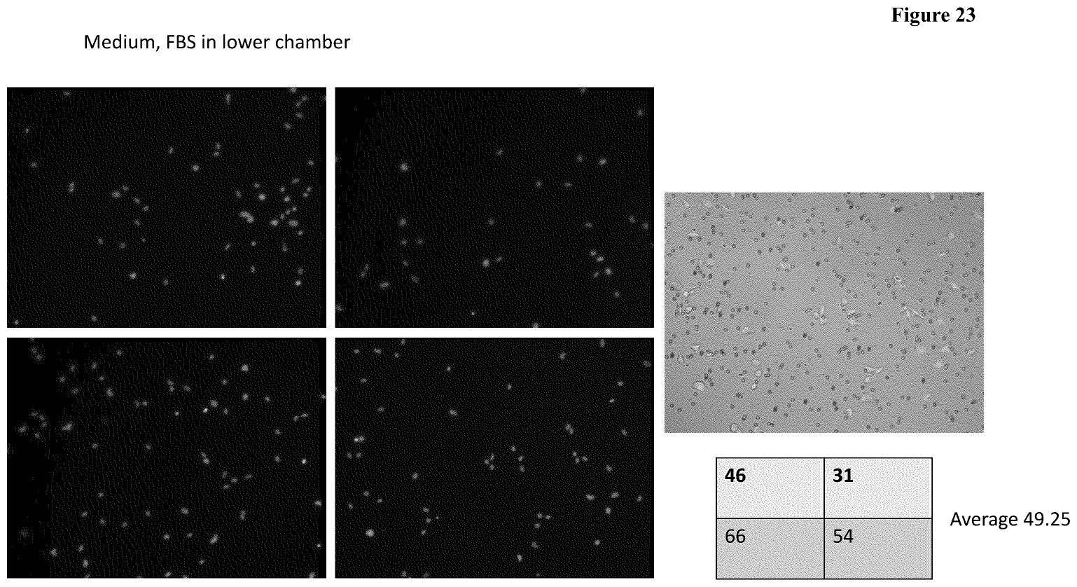

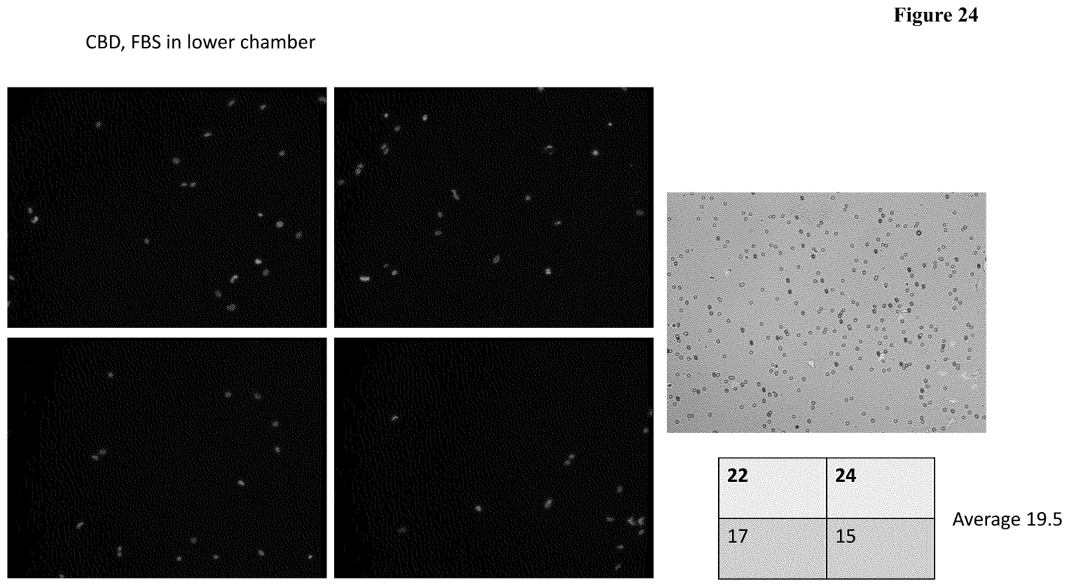

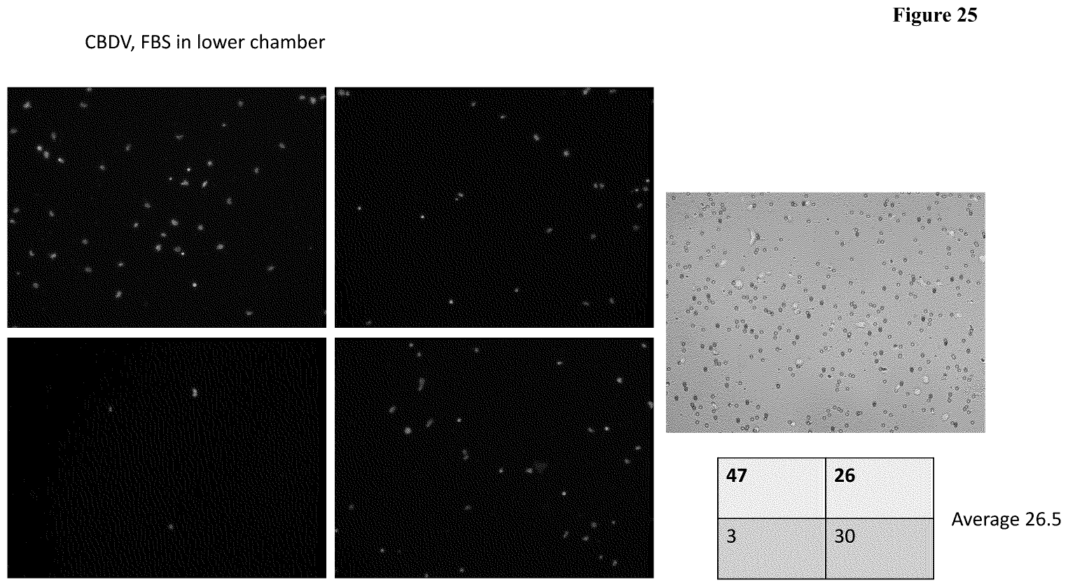

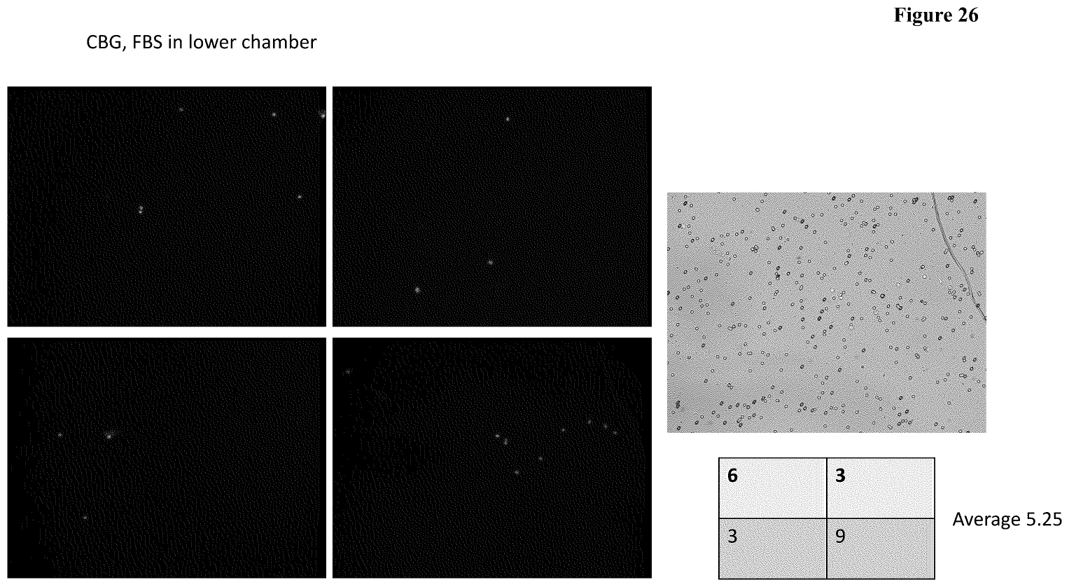

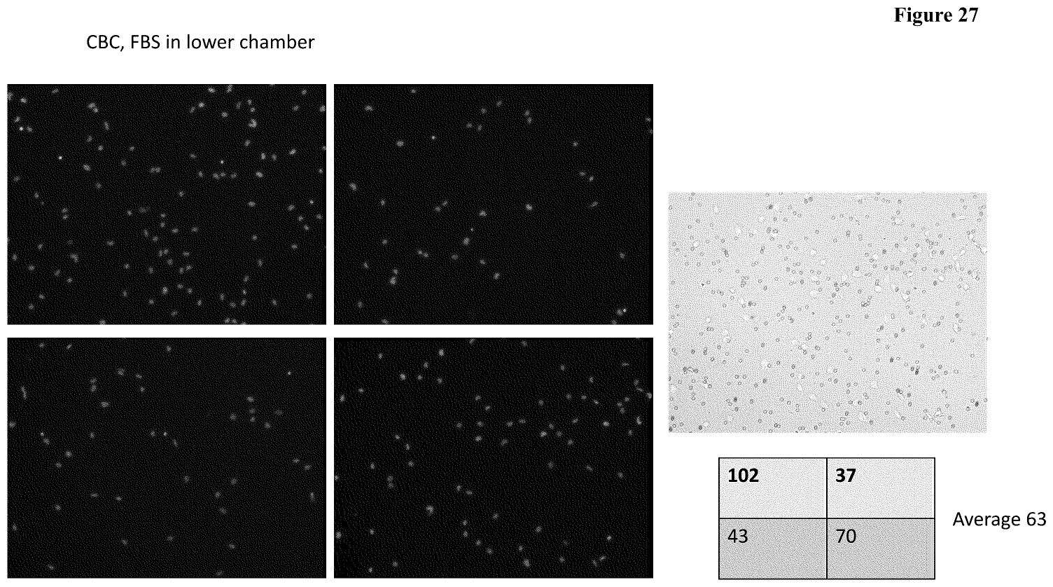

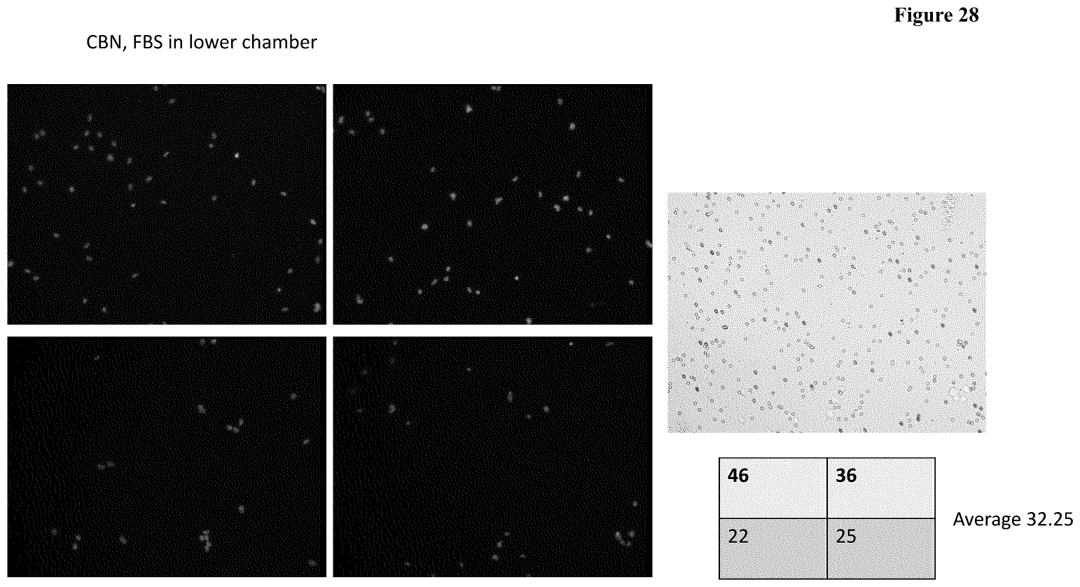

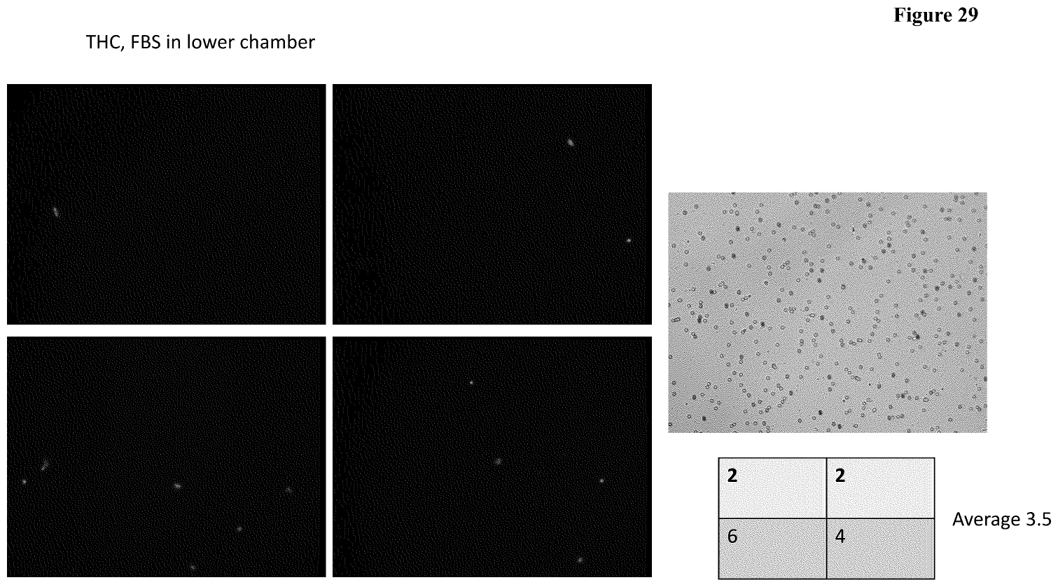

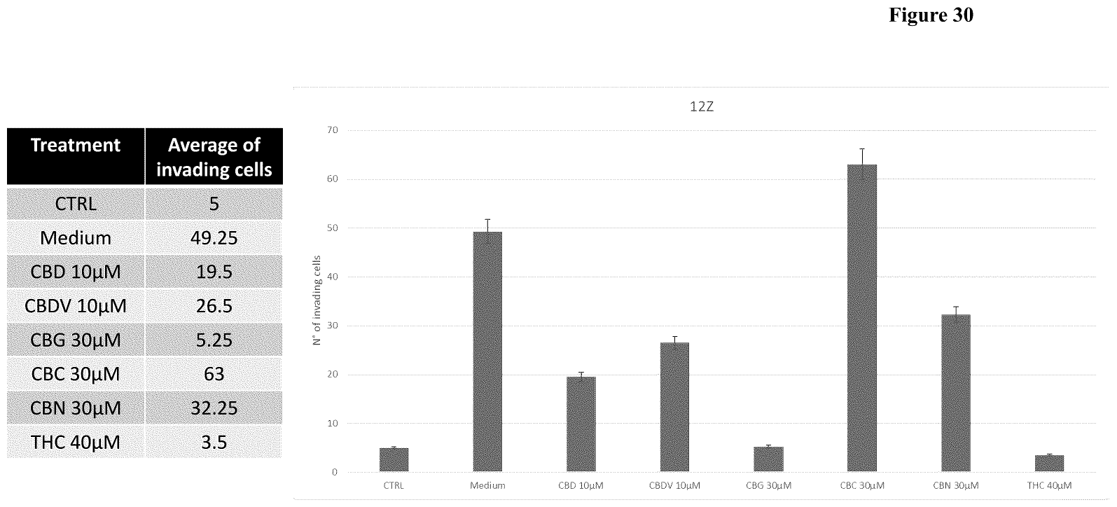

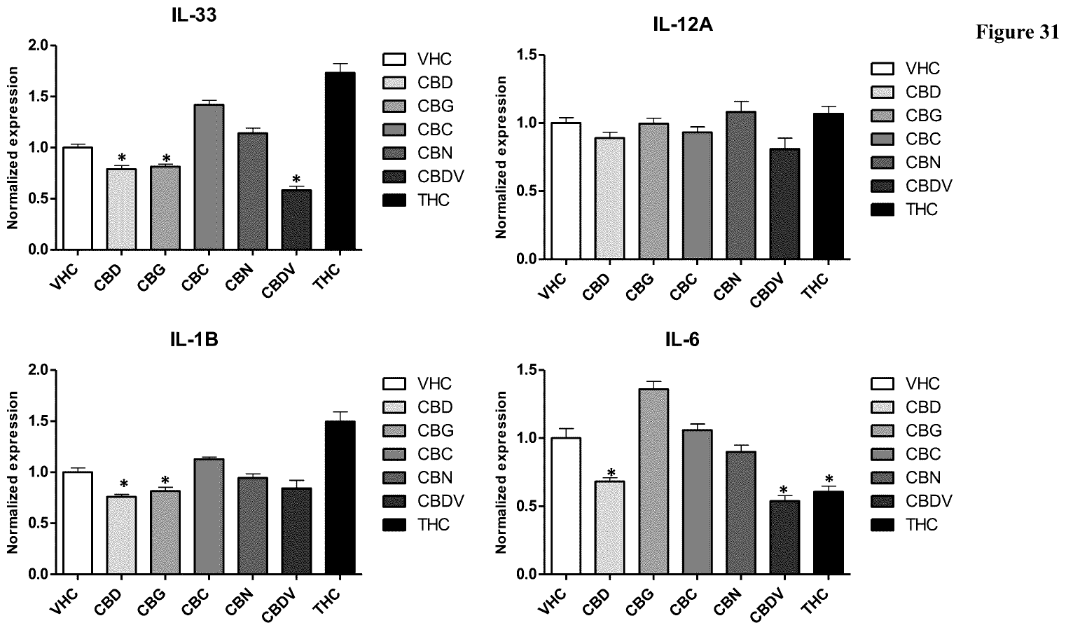

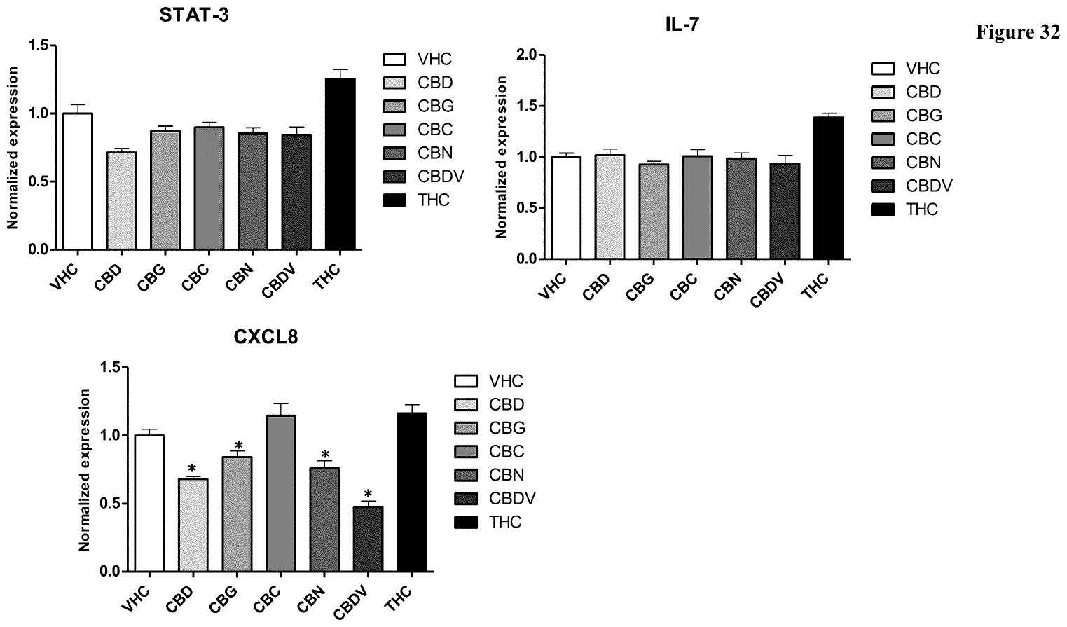

The cytotoxic effect was also evaluated in one human pancreatic ductal adenocarcinoma cell line (PANC-1) and in one glioblastoma cell line (U251). The results showed that the single phytocannabinoids were effective with different potency also in other human cancer cell lines, as shown (FIG. 11-14).

Endometriosis is a painful disorder in which tissue like the tissue that normally lines the inside of the uterus, the endometrium, grows outside the uterus. Endometriosis is considered a benign disease although, like cancer, it has the characteristic of being an invasive disease with cells that have an enhanced capacity to migrate and invade surrounding tissues. So, a potential successful therapeutical approach must consider a drug that is able to reduce endometriotic cell proliferation, invasion and the inflammatory phenotype. We first evaluated the cytotoxicity for each phytocannabinoids, as reported (FIG. 15-21). The results evidenced as each phytocannabinoids exert a cytotoxic activity at different doses (FIG. 21). Second, we assessed the effect of non-cytotoxic dose for each phytocannabinoid in regulating cell invasion, by Transwell invasion assay. As reported (FIG. 22-30), the phytocannabinoids were effective in reducing endometriosis cells invasion with major effect observed for THC>CBG>CBD>CBDV>CBN>CBC, as summarized in FIG. 30. Third, since endometriosis is characterized by an inflammatory phenotype, we analyzed the expression of different cytokines involved in endometriosis, by RT/PCR. The results showed that CBD reduced IL-33, IL-1beta, IL-6, CXCL8, STAT-3; CBG reduced IL-33, IL-1 beta, CXCL8; CBN reduced CXCL-8; CBDV reduced IL-33, IL-6, CXCL-8 and THC reduced IL-6 (FIG. 31,32)

So, summarizing phytocannabinoids exert with different potency a cytotoxic activity, an inhibition of invasion and an anti-inflammatory activity in endometriotic cells.

### Materials and Methods

### Cell Lines

Ishikawa is a well differentiated type I cell line, was purchased from Sigma Aldrich (Milan, Italy). Ishikawa cells were grown in EMEM medium (Lonza, Milan, Italy), supplemented with 5% fetal bovine serum (FBS), 2 mM/L of glutamine, 100 IU/ml of penicillin, and 100 mg of streptomycin. Primary EC cells PCEM004A, poorly differentiated mixed type I/II cell line, was grown in RPMI1640, supplemented with 20% FBS, 2 mM/L of glutamine, 100 IU/ml of penicillin, and 100 mg of streptomycin. Immortalized human endometriotic cell line (12Z) was generated by infecting cells, obtained from a 37-year-old female undergoing laparoscopy, with SV40 virus. This cell line was purchased by Applied Biological Materials Inc and was maintained in Prigrow III medium supplemented with 20% no-heat inactivated FBS, 100 IU/ml of penicillin and 100 mg of streptomycin. Human pancreatic ductal adenocarcinoma PANC-1 cell line was purchased by Sigma Aldrich (Milan, Italy) and cultured in DMEM high glucose medium (EuroClone, Milan, Italy) supplemented with 10% FBS, 2 mM L-glutamine, 100 IU/mL penicillin, 100 mg streptomycin and 1 mM sodium pyruvate. The human glioblastoma U251 cell line (grade IV), obtained from European Collection of Cell Cultures (ECACC, Salisbury, UK), was maintained in EMEM supplemented with 10% FBS, 2 mmol/L L-glutamine, 100 IU/mL penicillin and 100 μg streptomicin. Media were changed every 48 hours until cells were 90% confluent. All cell lines were maintained at 37 C with 5% CO₂ and 95% humidity.

### MTT Assay

EC cell lines, PANC-1, U251, and 12Z (3×10⁴ cells/ml) were plated in 96-well plates, in a final volume of 100 microl/well. After 24 hours, treatments or vehicles were added for 72 hours. At least six replicates were used for each treatment. At the indicated time point, cell viability was assessed by adding 0.8 mg/ml of 3-[4,5-dimethylthiazol-2-yl]-2,5 diphenyl tetrazolium bromide (MTT, Sigma-Aldrich) to the media. After 3 hours, the plates were centrifuged, the supernatant was removed, and the pellet was solubilized with 100 microl/well of DMSO. The absorbance of the samples against a background control (medium alone) was measured at 570 nm using an ELISA reader microliter plate (BioTek Instruments, Winooski, VT, United States). IC₅₀ was calculated by PRISM 5.0a software, for single phytocannabinoids.

### Invasion Assay

12Z cell invasion was evaluated by Transwell invasion assay using Transwell chambers (BD Biosciences, Franklin Lakes, NJ, USA) characterized by the upper face of the polycarbonate membrane covered with 1 mg/ml Matrigel. A total of 750 μl cell culture medium supplemented with 10% FBS was added in the lower chamber. Serum free culture medium (500 l) containing 2.5×10⁴ 12Z endometriotic cells was plated into the upper chamber with the appropriate treatment. After 24 hours of incubation, the cells remaining in the upper chamber were removed by cotton swabs. The cells on the lower and inner surface of the membrane were fixed in cold methanol, stained with hoechst and observed using a fluorescence microscope (BX51 fluorescence microscope, Olympus, Milan, Italy). A total of ten fields at ×10 magnification were selected at random to measure the average cell coverage using ImageJ software version 1.45s (National Institutes of Health, Bethesda, MD, USA). The experiments were performed in triplicate at least three times independently.

### RNA Isolation, Reverse Transcription and Quantitative Real-Time PCR Taqman Array

Total RNA from untreated or single treated 12Z cell line with CBD, CBG, CBC, CBN, CBDV and THC was extracted using Rneasy Mini kit (Qiagen, Milan, Italy). One microgram of total RNA from each sample was subjected to reverse transcription in a total volume of 20 μL using the High-Capacity cDNA Archive Kit (Applied Biosystem, Foster City, PA, USA) according to the instructions. cDNAs were analyzed by qRT-PCR performed using an IQ5 Multicolor Real time PCR Detection system. Quantitative real-time gene expression was performed with the TaqMan® Array Plate, containing 15 associated cytokine genes and beta-actin as endogenous control gene, was purchased (Thermo Fisher, Grand Island, NY, USA) and used to evaluate the treatments in modulating 12Z-related genes. Measurement of housekeeping gene (beta-Actin) on the samples was used to normalize mRNA content. The gene expression levels of treated cell lines were expressed as normalized fold compared with vehicle-treated cells.

### Statistical Analysis

Data presented as the mean values ±S.D./S.E. and are representative of the results of at least two independent experiments. The significance of the differences between data (**p<0.05) was assessed using Student's t-test or the one-way analysis of variance (ANOVA). The statistical analysis was performed using Prism 5.0a (Graph Pad).

### II. Methods and Uses

The phytocannabinoids delta9-tetrahydrocannabinol (THC), cannabigerol (CBG), cannabinol (CBN), cannabichromene (CBC) and cannabidivarin (CBDV) alone, and in combinations (comprising combination with CBD), induced cytotoxicity in endometrial cancer cell lines. The results suggested that at the appropriate doses all cannabinoids reduced cell viability.

The cell lines were also treated with phytocannabinoids to show treatment of endometriosis.

Accordingly, the present disclosure includes a method of treating endometrial cancer in a subject in need thereof, comprising administering an effective amount of at least one phytocannabinoid to the subject, with the proviso that the at least one phytocannabinoid does not comprise cannabidiol (as single treatment only). The present disclosure also includes a use of an effective amount of at least one phytocannabinoid for treatment of endometrial cancer in a subject in need thereof, with the proviso that the at least one phytocannabinoid does not comprise cannabidiol (as single treatment only). The present disclosure also includes a use of an effective amount of at least one phytocannabinoid for preparation of a medicament for treatment of endometrial cancer in a subject in need thereof, with the proviso that the at least one phytocannabinoid does not comprise cannabidiol (as single treatment only). The present disclosure also includes at least one phytocannabinoid for use to treat endometrial cancer in a subject in need thereof, with the proviso that the at least one phytocannabinoid does not comprise cannabidiol (as single treatment only).

In an embodiment, the at least one phytocannabinoid comprises, consists essentially of or consists of (or “is”) cannabidiol, delta9-tetrahydrocannabinol, cannabidivarin, cannabinol, cannabigerol, cannabichromene or combinations thereof. In another embodiment, the at least one phytocannabinoid comprises cannabidiol, delta9-tetrahydrocannabinol, cannabidivarin, cannabinol, cannabigerol, cannabichromene or combinations thereof. In a further embodiment, the at least one phytocannabinoid consists essentially of cannabidiol, delta9-tetrahydrocannabinol, cannabidivarin, cannabinol, cannabigerol, cannabichromene or combinations thereof. In another embodiment of the present disclosure, the at least one phytocannabinoid consists of (or “is”) cannabidiol, delta9-tetrahydrocannabinol, cannabidivarin, cannabinol, cannabigerol, cannabichromene or combinations thereof.

In an embodiment, cannabidiol is provided in combination with another phytocannabinoid to treat endometrial cancer.

Of the individual phytocannabinoids administered to endometrial cancer cell lines, the efficacy in reducing cell viability was generally highest for CBN. Accordingly, in an embodiment, the at least one phytocannabinoid consists essentially of or consists of (or “is”) cannabinol. In another embodiment, the at least one phytocannabinoid consists essentially of cannabinol. In a further embodiment, the at least one phytocannabinoid consists of (or “is”) cannabinol.

However, a dose dependent effect in all endometrial cancer cell lines was also observed for the other individual phytocannabinoids CBD, THC, CBDV, CBG and CBC tested in the present examples. Accordingly, in an embodiment, the at least one phytocannabinoid consists essentially of or consists of (or “is”) cannabidivarin. In another embodiment, the at least one phytocannabinoid consists essentially of delta9-tetrahydrocannabinol. In another embodiment, the at least one phytocannabinoid consists essentially of cannabigerol. In a further embodiment, the at least one phytocannabinoid consists of (or “is”) cannabinol. In an embodiment, the at least one phytocannabinoid consists essentially of or consists of (or “is”) cannabigerol. In another embodiment, the at least one phytocannabinoid consists essentially of cannabigerol. In a further embodiment, the at least one phytocannabinoid consists of (or “is”) cannabigerol. In an embodiment, the at least one phytocannabinoid consists essentially of or consists of (or “is”) cannabichromene. In another embodiment, the at least one phytocannabinoid consists essentially of cannabichromene. In a further embodiment, the at least one phytocannabinoid consists of (or “is”) cannabichromene.

The combination of CBG or CBC with CBN were surprisingly observed to have the highest efficacy in reducing cell viability followed by combinations comprising CBN and one of the other phytocannabinoids. Several of the particular doses for these combinations were observed to be more effective compared to the sum of the individual compounds.

Accordingly, in an embodiment, the at least one phytocannabinoid consists essentially of or consists of (or “is”) a combination of cannabigerol and cannabichromene. In another embodiment, the at least one phytocannabinoid consists essentially of a combination of cannabigerol and cannabichromene. In a further embodiment, the at least one phytocannabinoid at least one phytocannabinoid consists of (or “is”) a combination of cannabigerol and cannabichromene.

In an embodiment, the combination of at least one phytocannabinoid that is cannabidivarin, cannabinol, cannabigerol or cannabichromene is a combination comprising cannabinol. In another embodiment, the combination further consists of cannabigerol. For example, the at least one phytocannabinoid consists essentially of or consists of (or “is”) a combination of cannabinol and cannabigerol. In another embodiment, the at least one phytocannabinoid consists essentially of a combination of cannabinol and cannabigerol. In a further embodiment, at least one phytocannabinoid consists of (or “is”) a combination of cannabinol and cannabigerol. Alternatively, in a further embodiment, the combination further consists of cannabichromene. For example, the at least one phytocannabinoid consists essentially of or consists of (or “is”) a combination of cannabinol and cannabichromene. In another embodiment, the at least one phytocannabinoid consists essentially of a combination of cannabinol and cannabichromene. In a further embodiment, the at least one phytocannabinoid consists of (or “is”) a combination of cannabinol and cannabichromene. Alternatively, in another embodiment, the combination further consists of cannabidivarin. For example, the at least one phytocannabinoid consists essentially of or consists of (or “is”) a combination of cannabinol and cannabidivarin. In another embodiment, the at least one phytocannabinoid consists essentially of a combination of cannabinol and cannabidivarin. In a further embodiment, the at least one phytocannabinoid consists of (or “is”) a combination of cannabinol and cannabidivarin.

In an embodiment, the combination of at least one phytocannabinoid that is cannabidivarin, cannabinol, cannabigerol or cannabichromene is a combination comprising cannabigerol. In another embodiment of the present disclosure, the combination of at least one phytocannabinoid that is cannabidivarin, cannabinol, cannabigerol or cannabichromene is a combination comprising cannabichromene. In a further embodiment, the combination of at least one phytocannabinoid that is cannabidivarin, cannabinol, cannabigerol or cannabichromene is a combination comprising cannabividarin.

In an embodiment, the at least one phytocannabinoid consists essentially of or consists of (or “is”) a combination of cannabigerol, cannabinol and cannabidivarin. In another embodiment, the at least one phytocannabinoid consists essentially of a combination of cannabigerol, cannabinol and cannabidivarin. In a further embodiment, the at least one phytocannabinoid consists of (or “is”) a combination of cannabigerol, cannabinol and cannabidivarin. In another embodiment, the at least one phytocannabinoid consists essentially of or consists of (or “is”) a combination of cannabichromene, cannabinol and cannabidivarin. In another embodiment, the at least one phytocannabinoid consists essentially of a combination of cannabichromene, cannabinol and cannabidivarin. In a further embodiment, the at least one phytocannabinoid consists of (or “is”) a combination of cannabichromene, cannabinol and cannabidivarin. In another embodiment, the at least one phytocannabinoid consists essentially of or consists of (or “is”) a combination of cannabichromene, cannabinol and cannabigerol. In another embodiment, the at least one phytocannabinoid consists essentially of a combination of cannabichromene, cannabinol and cannabigerol. In a further embodiment, the at least one phytocannabinoid consists of (or “is”) a combination of cannabichromene, cannabinol and cannabigerol. In another embodiment, the at least one phytocannabinoid consists essentially of or consists of (or “is”) a combination of cannabichromene, cannabidivarin and cannabigerol. In another embodiment, the at least one phytocannabinoid consists essentially of a combination of cannabichromene, cannabidivarin and cannabigerol. In a further embodiment, the at least one phytocannabinoid consists of (or “is”) a combination of cannabichromene, cannabidivarin and cannabigerol.

In an embodiment, the at least one phytocannabinoid consists essentially of or consists of (or “is”) a combination of cannabigerol, cannabinol, cannabidivarin and cannabichromene. In another embodiment, the at least one phytocannabinoid consists essentially of a combination of cannabigerol, cannabinol, cannabidivarin and cannabichromene. In a further embodiment of the present disclosure, the at least one phytocannabinoid consists of (or “is”) a combination of cannabigerol, cannabinol, cannabidivarin and cannabichromene.

In an embodiment, the at least one phytocannabinoid is an individual phytocannabinoid. In another embodiment, the at least one phytocannabinoid is a combination of two phytocannabinoids. In a further embodiment, the at least one phytocannabinoid is a combination of three phytocannabinoids. In another embodiment of the present disclosure, the at least one phytocannabinoid is a combination of four phytocannabinoids.

In an embodiment, THC and THC combinations are provided with the at least one phytocannabinoid.

In an embodiment, the at least one phytocannabinoid is administered or for use in combination with at least one other anticancer treatment.

In an embodiment, the subject is a human.

The at least one phytocannabinoid is administered to a subject or used in a variety of forms depending on the selected route of administration or use, as will be understood by those skilled in the art. In an embodiment, the at least one phytocannabinoid is administered to the subject or used, for example, by enteral or parenteral routes, and the at least one phytocannabinoid formulated accordingly. Conventional procedures and ingredients for the selection and preparation of suitable compositions are described, for example, in Remington: The Science and Practice of Pharmacy (2020—23rd edition) and in The United States Pharmacopeia: The National Formulary (USP 43 NF 37) published in 2019.

Enteral administration or use includes all suitable routes involving the gastrointestinal tract, for example, oral, buccal, sublingual, nasal and rectal. In an embodiment of the present disclosure, the enteral administration or use of the at least one phytocannabinoid is oral administration or use; i.e. the at least one phytocannabinoid is administered orally or is for oral use, as the case may be. Formulations suitable for oral administration or use may be prepared by methods known to a person skilled in the art.

Parenteral administration or use includes intravesical, intravenous, intraperitoneal, subcutaneous, intramuscular, transepithelial, intrapulmonary, intrathecal, and topical modes of administration or use. Formulations suitable for parenteral administration or use may be prepared by known methods by a person skilled in the art.

Treatment methods comprise administering to a subject or use of an effective amount of the at least one phytocannabinoid and optionally consist of a single administration or use, or alternatively comprise a series of administrations or uses. For example, the at least one phytocannabinoid is administered or used at least once a week. However, in another embodiment, the at least one phytocannabinoid is administered to the subject or used from one time per three weeks or one time per week to once daily for a given treatment or use. In another embodiment, the at least one phytocannabinoid is administered or used 2, 3, 4, 5 or 6 times daily. The length of the treatment period depends on a variety of factors, such as the severity of the disease, disorder or condition such as the age and/or sex of the subject, and the activity and/or formulation of the at least one phytocannabinoid and/or a combination thereof. It will also be appreciated that the effective amount of the at least one phytocannabinoid used for the treatment or use may increase or decrease over the course of a particular treatment regime or use. Changes in dosage may result and become apparent by standard diagnostic assays known in the art. In some embodiments of the present disclosure, chronic administration or use may be required. For example, the at least one phytocannabinoid is administered or used in an amount and for a duration sufficient to treat the subject.

In embodiments, wherein the at least one phytocannabinoid is a combination of at least two phytocannabinoids, the phytocannabinoids are either used or administered separately in time and/or in mode of administration or use (i.e. different routes of administration or use) or they are administered or for use together in the same pharmaceutical preparation and/or at the same time, which may depend, for example, on the identity of the phytocannabinoids.

In an embodiment, the at least two phytocannabinoids are used or administered separately in time and/or in mode of administration or use.

In another embodiment, the at least two phytocannabinoids are administered or for use contemporaneously. As used herein, contemporaneous administration or use, for example, of two substances to a subject means providing the first phytocannabinoid and the second phytocannabinoid so that the pharmacological effects of the first phytocannabinoid and the second phytocannabinoid are present in the subject at the same time. The exact details of the administration or use will depend on the pharmacokinetics of the first phytocannabinoid and the second phytocannabinoid in the presence of each other, and can include administering or use of the first phytocannabinoid and the second phytocannabinoid within a few hours of each other, or even administering or use of the first phytocannabinoid and the second phytocannabinoid within 24 hours, or 48 hours or greater of administration or use of the other, if the pharmacokinetics are suitable. Design of suitable dosing regimens is routine for one skilled in the art. In some embodiments, the at least two phytocannabinoids are administered or used substantially simultaneously; i.e. within minutes of each other or in a single composition that comprises both substances. In another embodiment, the at least two phytocannabinoids are administered to a subject or for use in a non-contemporaneous fashion. In a further embodiment, the at least two phytocannabinoids are administered to a subject or for use in a contemporaneous fashion followed by, or alternating with, administration or use in a non-contemporaneous fashion.

The dosage of the at least one phytocannabinoid can vary depending on many factors such as the pharmacodynamic properties of the phytocannabinoid or combination thereof, the mode of administration or use, the age, health and weight of the subject, the nature and extent of the symptoms of the disease, disorder or condition such as the frequency of the treatment or use and the type of concurrent treatment or use, if any, and/or the clearance rate of the phytocannabinoid in the subject. One of skill in the art can determine the appropriate dosage having regard to the above factors. In an embodiment, the at least one phytocannabinoid is administered or used initially in a suitable dosage that is optionally adjusted as desired, depending on the clinical response. As a representative example, oral dosages of the at least one phytocannabinoid may range from less than 1 mg per day to 1000 mg per day for a human subject. In an embodiment of the present disclosure, the at least one phytocannabinoid is formulated in a pharmaceutical composition suitable for oral administration or use and the compounds are, for example, present in an amount of about 0.001, 0.01, 0.1, 0.25, 0.5, 0.75, 1.0, 5.0, 7.5, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per dose. In another embodiment of the present disclosure, the at least one phytocannabinoid is administered or used in a single daily dose or the total daily dose may be divided into two, three or four daily doses.

In an embodiment, cannabidiol is provided in combination with another phytocannabinoid to treat endometriosis.

In certain embodiments, the phytocannabinoids used to treat endometrial cancer are also applicable to treat endometriosis.

The following non-limiting examples are illustrative of the present disclosure:

### III. Results

Phytocannabinoids induce cytotoxicity in endometrial cancer cell lines, pancreatic ductal adenocarcinoma cell line, glioblastoma cell line:
Chemotherapeutic drugs+phytocannabinoids in cancer
Endometriosis and phytocannabinoids: cytotoxicity, invasion, inflammation.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION REFERENCES

1. Makker, A.; Goel, M. M. Tumor progression, metastasis, and modulators of epithelial-mesenchymal transition in endometrioid endometrial carcinoma: An update. Endocr. Relat. Cancer 2016, 23, R85-R111;
2. Sonoda, K. Molecular biology of gynecological cancer. Oncol. Lett. 2016, 1, 16-22
3. Arora, E.; Masab, M.; Mittar, P.; Jindal, V.; Gupta, S.; Dourado, C. Role of immune checkpoint inhibitors in advanced or recurrent endometrial cancer. Cureus 2018, 10, e2521
4. The Effects of Cannabidiol and Prognostic Role of TRPV2 in Human Endometrial Cancer. Marinelli O, Morelli M B, Annibali D, Aguzzi C, Zeppa L, Tuyaerts S, Amantini C, Amant F, Ferretti B, Maggi F, Santoni G, Nabissi M. Int J Mol Sci. 2020 Jul. 29; 21(15):5409
5. Shafrir A L, Farland L V, Shah D K, et al. Risk for and consequences of endometriosis: a critical epidemiologic review. Best Pract Res Clin Obstet Gynaecol 2018; 51:1-15;
6. The World Bank. Population ages 15-64 (% of population). 2017 (https://data.worldbank.org/indicator/SP.POP.1564.TO.ZS
7. Agarwal S K, Chapron C, Giudice L C, et al. Clinical diagnosis of endometriosis: a call to action. Am J Obstet Gynecol 2019; 220(4): 354.el-354.e12
8. Machairiotis N, Vasilakaki S, Thomakos N. Inflammatory Mediators and Pain in Endometriosis: A Systematic Review. Biomedicines. 2021 Jan. 8; 9(1):54.
9. Jorgensen, H.; Hill, A. S.; Beste, M. T.; Kumar, M. P.; Chiswick, E.; Fedorcsak, P.; Isaacson, K. B.; Lau_enburger, D. A.; Gri_th, L. G.; Qvigstad, E. Peritoneal fluid cytokines related to endometriosis in patients evaluated for infertility. Fertil. Steril. 2017, 107, 1191-1199.e2.
10. Li S, Fu X, Wu T, Yang L, Hu C, Wu R. Role of Interleukin-6 and Its Receptor in Endometriosis. Med Sci Monit. 2017, Aug. 5; 23:3801-3807.
11. Bulun S E. Endometriosis. N Engl J Med. 2009; 360:268-279

1. A method of treating an endometrial cancer in a subject in need thereof, comprising administering an effective amount of at least one phytocannabinoid to the subject, with the proviso that the at least one phytocannabinoid does not comprise cannabidiol in single administration.

2. The method of claim 1, wherein the at least one phytocannabinoid comprises cannabidivarin, delta9-tetrahydrocannabinol, cannabinol, cannabigerol, cannabichromene or their combinations with or without cannabidiol thereof.

3. The method of claim 1, wherein the at least one phytocannabinoid is cannabidivarin, cannabinol, delta9-tetrahydrocannabinol, cannabigerol, cannabichromene or their combinations with or without cannabidiol thereof.

4. The method of claim 1, wherein the at least one phytocannabinoid is cannabidivarin.

5. The method of claim 1, wherein the at least one phytocannabinoid is cannabinol.

6. The method of claim 1, wherein the at least one phytocannabinoid is a combination of cannabigerol and cannabichromene.

7. The method of claim 3, wherein the at least one phytocannabinoid is a combination comprising cannabinol.

8. The method of claim 7, wherein the combination further consists of cannabigerol.

9. The method of claim 7, wherein the combination further consists of cannabichromene.

10. The method of claim 7, wherein the combination further consists of cannabidivarin or delta9-tetrahydrocannabinol.

11. The method of claim 1, where the cancer is endometrial cancer.

12. The method of claim 1, wherein the at least one phytocannabinoid is administered in combination with at least one other anticancer treatment.

13. The method of claim 1, wherein the subject is a human.

14. The method of claim 1, wherein the effective amount of at least one phytocannabinoid to the subject is provided in a single dose.

15. The method of claim 1, wherein the effective amount of at least one phytocannabinoid to the subject is provided in a double dose.

16. The method of claim 1, wherein the effective amount of at least one phytocannabinoid to the subject is provided in a triple dose.

17. The method of claim 1, wherein the effective amount of at least one phytocannabinoid to the subject is provided in a quadruple dose.

18. The method of claim 1, wherein the concentration of at least one phytocannabinoid is up to 80 μM.

19. The method of claim 1, wherein the dose administered is from 0.001 to 1000 mg of active ingredient per dose.

20. A composition for treating an endometrial cancer in a subject in need thereof, comprising an effective amount of at least one phytocannabinoid, with the proviso that the at least one phytocannabinoid does not comprise cannabidiol in single administration.

21.-31. (canceled)
